(19) United States
(12) Patent Application Publication
(10) Pub. No.: US 2002/0098252 A1
(43) Pub. Date: Jul. 25, 2002

(54) Augmentation method for antitumor activity of substance containing amygdalin, composition containing augmented amygdalin contained substance, method for assessing antitumor efficacy of treatment for amygdalin contained substance and method for assessing antitumor substance containing amygdalin

(76) Inventor: Kozo Niwa, Tosashimizu-shi (JP)

Correspondence Address: JORDAN AND HAMBURG LLP, 122 EAST 42ND STREET, SUITE 4000, NEW YORK, NY, 10168, US

(21) Appl. No.: 10/038,428

(22) Filed: Oct. 24, 2001

Related U.S. Application Data
(62) Division of application No. 09/760,992, filed on Jan. 16, 2001.

(30) Foreign Application Priority Data
Jan. 17, 2000 (JP) 2000-8337

(51) Int. Cl. A61K 35/78; C12P 1/00

(52) U.S. Cl. 424/769; 424/776; 435/41

(57) ABSTRACT

The present invention is the invention for augmenting the anti-cancer efficacy in the amygdalin-contained loquat seed, etc. by roasting with infrared ray, fermenting with Koji, and oil-coat. The invention in relating to the increase peroxide lipid formation in the system by radiating the docosahexaenoic acid with ultra-violet ray under addition and increasing the concentration of treated product containing amygdalin from the processes. The more peroxide lipid formation, the stronger the anticancer efficacy in oral route is assessed and predicted. The invention in relation to apply the method of this invention to assess the efficacy of substance containing amygdalin.

### BACKGROUND OF THE INVENTION

This invention is related with techniques of augmentation method for antitumor activity of amygdalin belonging to glucoside contained in plant seeds assessment method, etc. especially with enlarging the expression of antitumor effect following oral route administration, or with predicting assessment of efficacy in antitumor agent following administration by oral route.

Loquat seed was known so far as to have antitumor effect and used in the fields of Kampo (Chinese medicine) and of health foods. There have been many research reports concerning so-called anti-tumor effect of loquat seed. Its principle composition for anti-cancer generally considered is amygdalin, a glucoside. In brief, loquat seed as a dried powder is administered orally when use for treatment of antitumor purpose.

On the other hand, whether the substances of plant origin possess their antitumor effects or not is proved by mouse test. Mouse groups are fed with diets with or without the extracted solution from plant. Comparative study of both groups is performed on assessing items, for example; extermination of tumor, reduce of tumor size or metastasis state, or survival rate of mice for proving that the antitumor effect is found with such animal test. Furthermore, toxicity in animal test and efficacy, safety tests in human beings are performed. After these data obtained, a clinical trial in medical facility is performed. Finally the efficacy of antitumor agent for human use is thus confirmed by clinical trial.

### SUMMARY OF THE INVENTION

Loquat seed responsible for carcinostatic and antitumor and anti-metastatic activities are broadly known so far and the efficacy is propagandized everywhere. Dried substance of loquat seed is powdered and administered orally or ingredient the alcoholic extract of loquat seed with other compositions such as vitamins in a product of health drinks and health foods or in moxibustion.

Loquat seed and leaf contains comparatively high content of amygdalin, and is considered to possess anti-cancer, anti-tissive, analgesic and so on effects.

As to antitumor effect, contrary to the expectation, only very few case of dried seed powder demonstrated effective in the oral route administration. Though the anti-cancer effect could not be denied, only very few cases are thought to be effective in antitumor activity.

While the present inventors studied on the reasons why somewhat personal difference of antitumor effect found in loquat seed powder case, etc. and the carcinostatic mechanism in humans is studied. The present inventors considered effective measures to be taken for all patients to receive the efficacy of loquat seed.

Furthermore, if such effective measures were considered to be principally taken by using a herb medicine, etc. containing amygdalin. Early assessment for their efficacy of antitumor agents containing amygdalin in the recipients administered orally is followed with then this medicine will be launched earlier in World. This is the urgent job to rescue the painful patients.

On the other hand, dried powder of nature products is proved to have antitumor activity in animal tests, but the expected efficacy cannot be often obtained in the case of administration in humans. During the course of the efficacy finding trial or this is not a standardized clinical trial, an administration system for this purpose is set up under a definite conditions and is controlled during the period of administration. A follow-up is necessary for this trial. Thus a very long time required till the results obtained for deciding that efficacy is proved or not in the oral administration in humans.

If a complex disease were on set in the patients who are the subjects in the clinical trial, they are to be excluded for emergency treatment and thus the efficacy to be proved from them become impossible. In connection with this, the fact that many cases unable to find the antitumor efficacy in the long term clinical trial.

It is not easy to continue the long-term clinical trial under a condition of keeping an efficacy finding ability. Under clinical trial, efficacy for anti-cancer product is exactly found. The target natural products used take a long time to obtain antitumor activity. It is necessary to establish the method supporting by technology enabling assess the antitumor activity in shorter days.

The present inventors considered the necessity to develop a method of predicting antitumor activity of substances containing amygdalin of loquat seed and instead of the clinical trail by administration in humans. In addition to this, antitumor activity of loquat seed was compared with that of substance from Kampo (Chinese medicine), etc. to prove the efficacy of antitumor objectively.

Because many known antirumor resources other than loquat seed such as Chinese medicine containing amygdalin are known. Thus, point of view regarding this topic is better to broaden amygdalin-contained resources toward natural products such as Chinese medicine etc.

The purpose of present invention is to obtain the fact expression of efficacy for antitumor substances containing amygdalin.

The other purpose of present invention is to predict the antitumor effect of substances containing amygdalin of loquat seed when administered orally in human.

The other purpose of present invention is to offer the compositions containing amygdalin capable of expression of antitumor activity.

The detailed description below is to show the previous described purposes, other purposes and characteristics for this invention.

The invention is an augmentation method applicable to the substances containing amygdalin comprising the features: roasting process using far infrared ray, fermentation process adding microorganism fermentation of the substance after roasting process. Augmentation of antitumor activity found as the feature possessed in the roasted and fermented substance containing amygdalin than those substances not roasted and fermented.

Succeeding to the previous fermentation process, oil-coat process was performed by adding the oil obtained from sesame to the roasted fermented substance to wrap the antitumor substance. This comprises the feature of this invention. The feature with previous substance containing amygdalin is the plant seed containing amygdalin. The feature with the previous loquat seed, a roasting process was performed by far infrared ray to raw seed

The previous substance having augmented antitumor activity comprises the feature of roasting process by far infrared ray, fermentation process adding microorganism to the substance after roasting process. Through these processes, the substances possess the feature of augmented antitumor activity than that of substance not roasted and fermented.

The invention is a method to assess the efficacy of antitumor activity of treatment for the amygdalin-contained substances. To the producing system of peroxide lipid by radiation with ultraviolet ray on the unsaturated fatty acids such as docosahexaenoic acid, etc., the treated amygdalin-contained were added. The more concentration of substance containing amygdalin, the stronger of treatment for increasing the ratio of peroxide lipid. The feature is involved stronger antitumor activity of such process for substances containing amygdalin.

The feature of previous substance containing amygdalin is the plant seed containing amygdalin. The feature of the previous loquat seed, a roasting process was performed by far infrared ray to the raw seed.

The invention is an assessing method for the efficacy of antitumor substance containing amygdalin not through the clinical trial method. The invention has the features to predict the antitumor activity of oral route administration to be stronger using substances containing amygdalin adding to a system in which peroxide lipid produced by radiating the unsaturated fatty acids such as docosahexaenoic acid, etc. with far infrared ray to increase the ratio of lipid in the antitumor substances. This comprises a feature of predicting the efficacy of antitumor in oral route for the products containing amygdalin.

The previous substance containing amygdalin possesses the feature that roasting process with far infrared ray and followed with a fermentation process with addition of microorganism. These processes are applicable to the augment technique of antitumor activity for the previous substance containing amygdalin than that of not roasted and fermented.

A feature of this invention is, that succeeding to the fermentation process, the fermented substance adding with the oil obtained from sesame roasted with far infrared ray to wrap the antitumor compositions.

Another features of this invention is that the previous substance containing amygdalin is amygdalin-contained plant seed, the previous described plant seed is loquat seed and the loquat seed is roasted in raw state with far infrared ray.

The present inventors confirmed through the studies on the inhibitory effects of anti-active oxygen substances in Kampo medicine (Chinese medicine) that various infectious diseases caused by and affected by active oxygen and peroxide lipid due to their destroying the tissues in human body.

An enzyme, its name called as superoxide dismutase (SOD) occurring in human body can remove the active oxygen to protect the body from active oxygen destroying cells. SOD content gradually decreases depending on age and at the level of SOD that cannot remove the active oxygen, especially the SOD level at the age more than 40s, the incorporated active oxygen in body cannot remove active oxygen. The irremovable active oxygen in the body caused the adult diseases.

And in the case that the active oxygen accumulated in the body due to a chronic and acute stimulations and chemical substances formed in human body, the quantity of SOD occurring in the body can not completely remove the active oxygen and thus leads to the on set of various diseases.

With this connection, those diseases caused by shortage of SOD and active oxygen cannot be completely removed, exogenous SOD administration is a relief to amend shortage as the measures taken for such case. This treatment is found to be effective only with injection route and no effect in oral route. The reasons why oral route administered SOD is not effective is left unexplained.

The present inventors ascertained that SOD is not effective when administered in oral route. Because it is instable against gastric juice and SOD their molecular weight more than 30,000 is not absorbed from digestive organ. There is a limit for SOD. It only removes the superoxide species (O₂⁻) among the four kinds of active oxygen species.

The present inventors studied on the substances possessing the ability to remove the over active oxygen and possible to administer by oral route and found that cereals such as rice, wheat etc., bean such as soybeans, red bean and their bean sprout can be processed by roasting and fermentation processes and then added with vegetable oils obtained from roasted plants to formulate an formulation as the effective product of removal for active oxygen in oral route (reference: Japanese Patent No.2125887).

The plant seeds and their sprouts originally contained the anti-active oxygen low molecules: flavonoids, polypherols, tannins, tocopherols, vitamins, B₂ etc. These anti-active oxygen low molecular weight compounds are bound each other to form the high molecular weight compound or molecular weight compounds. The present inventors found that intake of them as they are, the anti-active oxygen activity as expected from the low molecular weight compounds cannot be obtained.

Namely, the gastric juice of persons cannot degrade the active oxygen inhibiting high molecular substances into small molecule after intake. Almost patients cannot enjoy the effect of active oxygen inhibiting substances. The present inventors described this point in “Food Industry” journal Vol. 35, No. 14, entitled “Development, Improvement of DDS, SOD-like acting Foods for Natural Plant Seeds and Their Pharmacological Biochemical Discussions”.

The present inventors studied how the anti-active oxygen substances at their states containing in cereals and beans, etc. such seeds and sprouts can be activated and used for treatment and prevention of diseases.

As a result, under a mild heating, the active oxygen inhibiting substances in the plant seeds and their sprouts, part of low molecule compound can be set free from the complex state or part of complex molecule can be activated by chemical change and the functional group is put out. Then the anti-active oxygen activity is especially augmented through this heat treatment.

On the other hand, it has been considered that carcinostatic, antitumor or anti-metastasis activities of loquat seed are principally due to amygdalin. Amygdalin is different from the low molecular weight molecules having active oxygen inhibitory activity by chemical structure. Thus amygdalin was not the target compound for the serial studies relating to active oxygen.

But the present inventors obtained a finding that antitumor efficacy of loquat seed powder can be found in some patients, in contrast to this, another patients no efficacy, both patients administered by oral route. This finding can not be explained as the fact of substances following degradation to low molecule weight showing anti-active oxygen.

Namely, though the mechanism of antitumor activity of loquat seed was not elucidated, as described above, the expression of antitumor activity of loquat seed powder showed difference among the patients who were administered by oral route. This difference of expression for anti-tumor activity in patients may suggests that two type of patients. Those patients are easy to express the activity and another patients difficult to anti-tumor components.

After intake by oral route, loquat seed powder was sooner or later treated with gastric juice. Thus the patients are easy or difficult to express the antitumor activity were much dependent upon the treatment with gastric juice. Namely, there is a gastric juice treatment individual difference, this may be considered as a factor contributes to the difference of expression of anti-tumor activity.

Based on the clinical fact that difference in expression of anti-tumor activity due to individual strength and weakness of treatment with gastric juice, the present inventors submit a hypothesis on anti-tumor activity may be related with treatment of gastric juice as follows.

Namely, amygdalin showing antitumor composition contained in the loquat seed powder was the object treated by gastric juice. It exists in a state of difficult to express the antitumor activity and can be changed to the state easy to express the antitumor activity by treatment with gastric juice with individual differences.

While the patient with strong gastric juice in nature, expression of antitumor activity in the patients who were administered with loquat seed powder containing the antitumor composition: amygdalin. Thus expression of antitumor activity was proved.

But for the patients whose gastric juices were inborn weak and difficult to find the activity thus as a result, extent for expression of active was not so easy as that of patients with strong gastric juice.

It is considered and explained that many nowadays persons, their gastric juices are not strong enough to degrade the antitumor amygdalin contained in loquat seed powder and thus after intake of powder as it is, its efficacy found is very few.

Namely, patients their gastric juice is inborn strong, can solve the chain fastened (figuratively description) of the inactive state of composition of antitumor substance contained in the loquat seed. This means that it is existed in inactive form and gastric juice can degrade it into an active and free form. This leads to the expression of antitumor activity is lucidity.

It is considered that nowadays persons, their gastric juices can degrade the substance to a state of easy to express antitumor activity. For this purpose, effective compositions against tumor, their active sites are bound each other and thus in inactive state absorbed by intestine. This is the explanation why antitumor activity was not found in the patients.

In connection with this, the present inventors considered that if some treatment could be performed to convert the inactive form to active form for loquat seed, the above problem is solved by this idea.

But the present inventors cannot obtain the effective treating method in spite of several trials to convert the inactive form to active form. This is also due to one fact what states of active composition in inactive form in loquat seed, their complete findings were not obtained, etc. because of so complicated system was used.

It is out of their expectation that the present inventors had performed various experiments and at the final stage, the activation method for anti-active oxygen substances such as flavonoids, polypherols, etc. which are utterly different from amygdalin, was applied to amygdalin and found that conversion of amygdalin to active form is successful.

This activation method is applied to flavonoids which are utterly different from amygdalin and thus the inactive state is different from that of amygdalin. At the beginning, it is considered that application of activation method of anti-active oxygen substance to amygdalin is so much difficult.

As the results, it is out of the present inventors' expectation, activation method of anti-active oxygen substance is effective when apply to change the amygdalin, its structure different from anti-active oxygen substance to active state.

Namely, in the experiments, the present inventors use the roasted loquat seed powder prepared by far infrared ray radiation, showed the increased efficacy of antitumor in oral route when compared with that of not roasted loquat seed powder. Furthermore, to the roasted loquat seed powder, microorganism especially koji was inoculated and the fermentation was followed with. The antitumor activity was found to increase. This fact makes understanding by the present inventors.

It is known that those plant seeds in addition to loquat seed containing amygdalin and showing anti-tumor activity, for example in the seeds of apricot, peach, plum, Japanese apricot, Chinese peach, Amygdala amara. Furthermore, loquat leaf, alpha-alpha, bamboo shoot, etc. also contain comparative quantity of anti-cancer substances. Among these, the materials containing amygdalin are used in this invention.

No apparent effect is obtained from the dried loquat seed treated with roasting by far infrared ray and fermentation under various conditions. Thus it is better to use the seed in raw state.

Oil-coat after roasting with far infrared ray and fermentation contributes to increase of antitumor activity. This is to use the oil obtained from the sesame roasted with far infrared ray. Oil-coat is performed by wrapping the substance after fermentation.

As described above, the effective method to degrade the anti-active oxygen substances to low molecular weight for expression of antitumor activity can be applied to the case of amygdalin, that is a nitryl glucoside, different from the anti-active oxygen substances. This is out of the estimation by the present inventor at first. The method applied to amygdalin and this leads to the present invention.

Due to performing anti-cancer effect of loquat seed, after treated with the previous treating procedures, the substance became possessing extent of anti-tumor effect as high as that of Agaricus blazei, the latter has been known as attractive as for its antitumor effect.

Namely, the latent anti-cancer activity containing in amygdalin of loquat seed was not taken out completely. Thus its effect of antitumor was not proved. This can be approached by treatment of this invention, and the antitumor effect was appeared which could not be expected.

If the augment method for antitumor activity were applicable, those expensive rare crude drugs such as mushrooms, etc. will not be used and the cheap and conventional fruit seeds can be used as materials for development of medicine, health foods, etc. Decreasing economic burden of patients due to not use the expensive rare crude drug and use those materials easy to obtain. When consider from this point of view for the patients, the present invention is meaningful.

The substances containing amygdalin of loquat seed, etc. applied to the augmentation method for antitumor activity, when compared with the conventional substances only ingredient with dried powder from substances containing amygdalin, its antitumor effect was specially proved to be high.

Furthermore, during the serial researches performed, to the system of producing peroxide lipid from docosahexaenoic acid, etc. by radiation with ultraviolet ray, gradually increased the concentration of loquat seed powder treated with various processes, then the increased product of peroxide lipid at each concentration was found to increase dependent upon the concentration of loquat seed powder and antitumor activity was found to be stronger.

To the peroxide lipid produce system, same concentration of seed powder treated with various processes was added. It was successful to get the antitumor effect expressed by oral route for the product of large quantity production of peroxide lipid by determining the peroxide lipid content.

If these processes were adopted, even administration route is not oral, the finding ability for antitumor effect expressing in oral route can be predicted by what treatment performed for the substances containing amygdalin in loquat seed, etc. Even patients had not received the clinical trial, the antitumor efficacy in oral route can be used as the assessing method for antitumor efficacy.

Furthermore, a judgment was performed in unsaturated fatty acids such as docosahexaenoic acid can be derived to peroxide lipid by radiation with ultraviolet ray. To this system, substances containing amygdalin was added gradually by increasing concentration and peroxide lipid produced was measured. The stronger antitumor activity in oral route, the higher increment of peroxide lipid to which amygdalin added. Even not performing the clinical trial, comparison was able to perform easily for antitumor activity of substance containing amygdalin.

Furthermore, for example, to the production system of producing peroxide lipid by radiation on unsaturated fatty acids such as docosahexaenoic acid, the substances containing amygdalin possessing antitumor activity such as mushrooms, etc. and other category Chinese medicine was added individually, and gradually increased the concentration of substance added. By comparing the content of peroxide lipid formed and lining the order of content of peroxide lipid, augmentation of antitumor activity in oral route for the substances were easily compared to each other.

Namely those clinical trial not having the complete control for individual difference the comparison on relative strength of antitumor of substances in different category can be performed easily.

As the substances containing amygdalin, many plant and animal resources. can be used broadly. Crude drugs (Chinese medicine) using wild grown plants containing amygdalin, and those substances containing amygdalin of harvested materials from water fertilization cultivation of artificial cultivation can also be used. By applying this invention to the natural product or cultivated product and successful expression of antitumor was obtained. This can be used for those substances described in this invention as compositions containing amygdalin.

### DESCRIPTION OF THE PREFERRED EMBODIMENTS

The detailed explanation of the embodiments of this invention is shown below with several examples.

Augmentation method for antitumor activity substances containing amygdalin, compositions containing augmented amygdalin-contained substance, method for assessing antitumor efficacy of treatment for substances containing amygdalin and method for assessing antitumor efficacy of substances containing amygdalin using loquat seed as the substance containing amygdalin, were described below.

(EMBODIMENT 1)

The embodiment consists of augmentation method for antitumor activity of substance containing amygdalin and anti-cancer activity augmented product containing amygdalin-contained substance.

The augmentation method for substance containing amygdalin is firstly, roasting the loquat seed containing amygdalin with far infrared ray and followed with fermentation adding microorganism such as Koji(Aspergillus Oryzae) under definite conditions of humidity and temperature. After finish of fermentation, substance was ground into powder. The micro-powder was further oil-coated with the oil obtained from pressing the sesame roasted by far infrared ray.

Furthermore, to the loquat seed containing amygdalin, processed gradually by roasting with far infrared ray, fermentation with microorganism, oil-coating. The composed substances finally obtained become the substances showing antitumor activity in oral route.

By showing with Examples below, the detailed description of this invention was explained. Substances containing amygdalin that was used in this invention is loquat seed. According to the experiment, the state of seed used was in raw state. The raw state showed a stronger antitumor activity than that of dried one.

The first procedure for augmentation method of antitumor in the Example is the process of roasting by radiation with far infrared ray, by using the 4-14 μm wavelength far infrared ray. The inner side of far infrared ray with this wavelength radiating ceramic-coated vessels such as pan, kettle, etc. or vessels made of metallic oxide materials such as stones, ceramic soils, gravels and sands mixed with ceramic soils radiating far infrared ray are used for roasting.

The above described loquat seed was put into the vessel, for example, far infrared ray emitting materials such as granite, ceramics, celestite (TENSYOUSEKI), etc., radiation for 30-90 minutes till the state of material not charred under a condition of slow agitation and roasting.

The roasting method, not necessary to limit the extent that stimulating amygdalin can be set free from loquat seed.

Following to the roasting process with far infrared ray, the fermentation process was prepared as the second process. In this way, after treating the material with far infrared ray, fermentation with microorganism such as Koji under 30-36° C. with moisture was continued for 48-72 hours. By using a fermentation machine is permissive, the time required can be shortened.

The microorganisms other than Koji can be used in the fermentation. And during fermentation, in place of koji, the ripened papaya, pineapple juice, fig skin of grape, young bamboo was used.

Furthermore, digestive enzymes such as diastase, pancreatin, protease, pepsin, trypsin etc., microorganism originated protease, glucosidases for degradation of polysaccharide such as hemicellulase, etc. or precursors, etc. for the digestion enzymes and protease are used in fermentation process.

The present inventors found that among fermentation using microorganisms, fermentation using Koji showed a better augmentation for antitumor activity than other. And after fermentation the product was ground into powder.

Machine required for grinding the material into powder, the crasher sold in market was used but avoiding from heat during grinding is necessarily done. In order to avoid heat, a stone mill is used instead of machine crasher.

Thus the loquat seed material after far infrared ray radiation roast, fermentation process, was subjected to oil-coat process. The oil-coat was a process to wrap the micro-powder material after fermentation by using the oil obtained from sesame roasted with far infrared ray.

The oil used in the oil-coat process was the oil obtained from roasted sesame (abbreviated as “sesame paste oil” hereinafter). Sesame paste oil was prepared by pressing the roasted sesame which was slowly roasted by far infared ray its temperature not exceeding 100° C. Such sesame oil showed a paste appearance because micro-solids were left during crashing.

Adding the loquat seed material after fermented to the sesame paste oil and ground into micro-powder, the sesame paste oil was added to wrap the loquat seed micro-powder with sesame oil. Sesame oil showed a stronger osmotic force to the cells at the diseased portion than that of no oil-coat. Such oil-coat has been attracted recently from the viewpoint of drug delivery system (DDS). And this is an important process having the efficacy direction.

Carcinogen and virus, etc. first adhere to the cell membrane and grew there, and destroyed the cell membrane. The cell membrane is a place with full fats. Drugs including chemical reagents dissolved in water were rejected by membrane before approaching to it. But in this loquat seed material, it can approach to the membrane easily by this oil-coat process and this can be the factor contributes to antitumor effect.

As described above, oil-coat is an important process. Though it is better to set up this process, in some cases that oil-coat is not successfully performed, the oil-coating process can be omitted. Because as described below, even omitted the oil-coat process, if the far infrared ray radiation and fermentation processes have been exactly performed, augmenting the efficacy of antitumor was great. This experiment has been done for proof.

During the oil-coat, it is better to use mixture oil consisting of sesame paste oil described above and the oil collected from raw sesame by proper ratio.

In order to minimize particle size of oil drops, oil obtained from raw sesame is mixed with the oil from sesame paste oil and the viscosity increased for the mixture of oil. This leads to raise the osmotic power to target cell than that only coated with sesame paste oil its drop size bigger.

The oil collected from the raw sesame obtained by crashing the sesame and after mechanic pressing, the residual portion after removal of solid is the oil corresponding to the oil sold in the market.

Regarding the ratio for mixture of sesame paste oil described above and the raw sesame oil, for example, ratios 1:1 to 3 by weight is suitable. As to the rate of mixed oil to the micro-powder materials, for example, 1:4 to 5 ratios are better.

To the system of producing the peroxide lipid by radiation with ultraviolet ray to docosahexaenoic acid, the loquat seed its antitumor activity has been augmented as described above were added and the peroxide lipid quantity formed was measured. In connection with this, the efficacy of antitumor effect in oral administration for this product was investigated by clinical trial.

For the purpose to compare the augmentation methods described above, the loquat seed only treated with roasting by radiation of far infrared ray, the loquat seed only treated with fermentation with Koji, loquat seed treated with both far infrared ray radiation and fermentation with Koji, loquat seed treated with radiation, fermentation with Koji and oil-coat, the loquat seed not treated, were investigated on quantity of peroxide lipid formed, efficacy in clinical trial in oral administration. The raw state loquat seed was used in the treatments and for the purpose of comparison, roasted with far infrared ray, fermented with Koji and oil-coated loquat seed was prepared and the dried loquat seed was also used.

In the individual case of loquat seed only treated with roasting of far infrared ray, only treated with Koji fermentation, treated with both roasting and fermentation, micro-powder for each materials at its ending of treatment was prepared by the previous procedures for using as samples in the following experiments. The extent of grinding was kept at the same level of mean crashing particle size to remove effect from particle size.

Measurement for the quantity of peroxide lipid formed was performed below. To the testing tubes, added with a quantity of docosahexaenoic acid, each different quantity of powder of treated loquat seed described above was added and agitated. The mixture of each tube was subjected to ultraviolet radiation and peroxide lipid formed was measured.

Docosahexaenoic acid original solution was diluted with ethanol to ten-fold solution and further diluted to 200-fold solution. Sample solution of treated loquat seed (also non-treated one was prepared), was prepared at 60 mg/ml by adding ethanol and allowed to stand at room temperature for 2-6 weeks for ethanol extraction. Before measurement, the final concentration of extracted solution was adjusted at 0.6 mg/ml and used.

To 0.05 ml-portion of docosahexaenoic and diluted solution prepared according to the procedure described above. Sample 0.1 ml and ethanol 0.85 ml were added and mixed to a total volume of 1 ml. The mixture was radiated with ultra-violet ray for 3 hours to produce the peroxide lipid. The peroxide lipid formed was measured with TBA (Thiobarbituric Acid Reaction) method.

The resulting peroxide lipid was heated under acidic condition, the separated malondialdehyde (MDA) was reacted with thiobarbituric acid (TBA). The substance expressing maximum absorption was quantified and used to calculate the quantity of peroxide lipid indirectly.

To the TBA reaction mixture, 0.2 ml of 7% dodecyl sodium sulfate, 2 ml of 0.1 N hydrochloric acid, 0.3 ml of tungsten phosphoric acid, 1 ml of mixture solution of 0.67% TBA and acetic acid (1:1 by volume), were added and allowed to stand at 95° C. for 12 minutes for TBA reaction performance.

After cooling to room temperature, the TBA reaction layer was extracted with n-butyl alcohol and the n-butyl alcohol layer was measured by using fluorescence spectrometry for content of MDA. In the measurement Hitachi fluorescence spectrophotofluorometer, Type F 2000 equipped with 400 V photomultiplier was used, and fluorometry was carried out at 553 nm using the exciting light source of wavelength 515 nm.

Effect in the system of peroxide lipid production by radiation, docosahexaenoic acid with ultra-violet ray under various treatments are shown in Table 1 with value measured for MDA (equals to density of absorbance) by the method described above.

In connection with this, the individual product was used in clinical trial under time 3 g and 3 times a day administration in the patients with breast stomach and lung cancers, respectively The product covers untreated Koji fermentation for infrared roasting+Koji fermentation, far infrared roasting+Koji fermentation+oil-coat processed loquat seed. The results are shown in Tables 2, 3, and 4.

Table 2 demonstrates the efficacy of loquat seed treated with various methods described above in the patients with breast cancer. Table 3 and 4 demonstrates the efficacy of loquat seed treated with same processes in the patients with stomach cancer and lung cancer as that of Table 2. By using the formula the Rate of efficacy (%)=(effective+fairly effective) cases×100 (effective+fairly effective+not effective total cases).

The judgments of cases in Tables 2-4 were performed by comparison of tumor markers and size of shadow in photo diagnosis at the stage before and three months after oral administration of patients with cancer. The marker or the shadow size changed to size 40% original size in judged as “Effective” in the case of between more than 20% and less than 40% are judged as “Fairly Effective”

TABLE 1
Effect of Various Treatments on MDA Value

| | Samples | |
|---|---|---|
| | Control (Without Loquat | Mean MDA Value |
| Treatment | seed) | 115 |
| Non-Treatment | Raw loquat seed | 209 |
| Roast with Far | ″ | 320 |
| Infrared Ray | | |
| Fermentation with | ″ | 334 |
| Koji | | |
| Roasted with Infrared | 621 | |
| Ray + | | |
| Koji Fermentation | | |
| Roast with Far | ″ | 678 |
| Infrared Ray + Koji | Dried loquat seed | 572 |
| Fermentation + | | |
| Oil-coat | | |

Sample concentration: Added with 6 mg/ml of loquat seed treated with various processes.

TABLE 2
Efficacy of various treatments in the patients
with breast cancer

| | | Patients with Breast Cancer (75) | | | Rate of |
|---|---|---|---|---|---|
| | | | Fairly | Not | Efficacy |
| Treatment | Samples | Effective | Effective | Effective | (%) |
| Non-Treatment | Raw | 1 | 1 | 13 | 13.3 |
| | loquat | | | | |
| | seed | | | | |
| Koji Fermentation | Raw | 2 | 2 | 11 | 26.6 |
| | loquat | | | | |
| | seed | | | | |
| Roast with Far | Raw | 3 | 2 | 10 | 33.3 |
| Infrared Ray + | loquat | | | | |
| Koji Fermentation | seed | | | | |
| Roast with Far | Raw | 4 | 3 | 8 | 46.6 |
| Infrared Ray + | loquat | | | | |
| Koji Fermentation + | seed | | | | |
| Oil-coat | Dried | 2 | 1 | 12 | 20.0 |
| | loquat | | | | |
| | seed | | | | |

( ) = Indicates the total number of patients
$Rate of Efficacy = \frac{( Effective + Fairly effective ) cases}{Total Cases} \times 100 ( \% )$

TABLE 3
Efficacy of various treatments in the patients
with stomach cancer

| | | Patients with Stomach | | | |
|---|---|---|---|---|---|
| | | Cancer (40) | | | |
| | | | Fairly | Not | Rate of |
| | | Effec- | Effec- | Effec- | Efficacy |
| Treatment | Samples | tive | tive | tive | (%) |
| Non-Treatment | Raw loquat | 0 | 1 | 7 | 12.5 |
| | seed | | | | |
| Koji Fermentation | Raw loquat | 1 | 0 | 7 | 12.5 |
| | seed | | | | |
| Roast with Far | Raw loquat | 2 | 0 | 6 | 25.0 |
| Infrared Ray + | seed | | | | |
| Koji Fermentation | | | | | |
| Roast with Far | Raw loquat | 2 | 2 | 4 | 50.0 |
| Infrared Ray + | seed | | | | |
| Koji Fermentation + | Dried loquat | 1 | 1 | 6 | 25.0 |
| Oil-coat | seed | | | | |

( ) = Indicates the total number of patients

TABLE 4
Efficacy of various treatments in the patients
with lung cancer

| | | Patients with lung | | | |
|---|---|---|---|---|---|
| | | Cancer (60) | | | |
| | | | Fairly | Not | Rate of |
| | | Effec- | Effec- | Effec- | Efficacy |
| Treatment | Samples | tive | tive | tive | (%) |
| Non-Treatment | Raw loquat | 0 | 2 | 10 | 16.6 |
| | seed | | | | |
| Koji Fermentation | Raw loquat | 1 | 2 | 9 | 25.0 |
| | seed | | | | |
| Roast with Far | Raw loquat | 2 | 2 | 8 | 33.3 |
| Infrared Ray + | seed | | | | |
| Koji Fermentation | | | | | |
| Roast with Far | Raw loquat | 3 | 2 | 7 | 41.6 |
| Infrared Ray + | seed | | | | |
| Koji Fermentation + | Dried loquat | 2 | 1 | 9 | 25.0 |
| Oil-coat | seed | | | | |

( ) = Indicates the total number of patients

As shown in Table 1. The parameter for the production of peroxide lipid, MDA values are higher for samples treated with far infrared ray Koji fermentation and former oil-coat than that for sample treated with both roasting with far infrared ray and Koji fermentation. MDA values are larger for samples treated with for infrared ray and Koji fermentation than that for sample only treated with Koji fermentation. MDA values are higher for samples treated with Koji fermentation than that for samples treated with far infrared ray. MDA values are larger for the samples treated with for infrared ray than that for not treated.

This is to say, among them, the highest MDA values are obtained from the samples treated with far infrared ray, Koji fermentation and oil-coating. In the case of using dried seed as sample, MDA value is smaller than that of raw seed treated with far infrared ray and Koji fermentation. Even same treatments are applied, MDA value is higher for raw seed than that for dried seed.

On the other hand, clinical trial results are indicated in Tables 2-4 for patients with breast cancer, stomach cancer and lung cancer individually using product prepared from raw and dried loquat seeds. Rate of efficacy is higher for products from raw seed treated with Koji fermentation than that of no treatment. Rate of efficacy is higher for product from seed treated with roasting with far infrared ray and Koji fermentation than that of product from seed only roasted with far infrared ray. Rate of efficacy is higher for products from seed treated with roasting with far infrared ray, Koji fermentation and oil-coat than that for product from seed treated with roasting with far infrared ray and Koji fermentation.

For example, rate of efficacy of breast cancer for product not treated is 13.3% as shown in Table 3. In contrast to this, rate of efficacy for product of raw loquat seed treated with far infrared ray, Koji fermentation and oil-coat is 46.6%. This indicates that rate of efficacy has been increased to approximately 3.5-fold. On the other hand, when used the product from dried seed only increased approximately 1.5-fold.

Rate of efficacy of stomach cancer is shown in Table 3. The rate efficacy of sample from raw seed treated with far infrared ray, Koji fermentation and oil-coat increased 4-fold of raw seed not treated. Rate of efficacy of raw seed is higher by 2-fold than that of dried seed.

Rate of efficacy of lung cancer is shown in Table 4. The rate of efficacy of sample from raw seed treated with far infrared ray, Koji fermentation and oil-coat increased approximately 2.5-fold of the raw seed not treated, and the rate of efficacy of raw seed is approximately 1.7-fold of the dried seed.

These results strongly indicate that the processes of far infrared ray roasting, Koji fermentation and oil-coat incorporated gradually is effective to enlarge the efficacy of antitumor activity in oral route when the processes are applied to loquat seed.

Though the exact carcinostatic mechanism is left unexplained at the present stage and continuous study is required. The present inventors consider that non-treated loquat seed contains amygdalin having antitumor activity is fastened by chain (a figuratively expression) under an inactive state. The patient administered with this loquat seed, gastric juice of general person cannot destroy the chain and thus expected extent of antitumor activity is not found. Thus the treatment described above may suggest that the method of treatment contributes to activate amygdalin to active state and finally enlarge the antitumor efficacy.

TABLE 5
Comparison of MDA values of loquat seed with mushrooms

| | | | Mean |
|---|---|---|---|
| | | | MDA |
| | | Samples | Value |
| Treatment | Rank | Control (Not Added) | 115 |
| No Treatment | 1 | Grifola umbellate(CHYOREIMAITAKE) | 439 |
| | 2 | Grifola frondosa(MAITAKE(BLACK)) | 398 |
| | ★3 | Agaricus blazei | 283 |
| | 4 | Pachyma hoelen(BUKURYO) | 269 |
| | 5 | Tricholoma matsutake(MATSUTAKE) | 218 |
| | ☆6 | Raw loquat seed | 209 |
| | 7 | Lentinus edodes(SHIITAKE) | 189 |
| Roasted with | 1 | Grifola umbellate(CHYOREIMAITAKE) | 536 |
| Far Infrared | 2 | Grifola frondosa(MAITAKE(BLACK)) | 490 |
| Ray | ★3 | Agaricus blazei | 341 |
| | ☆4 | Raw loquat seed | 320 |
| | 5 | Pachyma hoelen(BUKURYO) | 288 |
| | 6 | MATSUTAKE | 271 |
| | 7 | Lentinus edodes(SHIITAKE) | 233 |
| Fermentation | 1 | Grifola umbellate(CHYOREIMAITAKE) | 594 |
| with Koji | 2 | Grifola frondosa(MAITAKE(BLACK)) | 528 |
| | ☆3 | Agaricus blazei | 399 |
| | ★4 | Raw loquat seed | 334 |
| | 5 | Tricholoma matsutake(MATSUTAKE) | 302 |
| | 6 | Pachyma hoelen(BUKURYO) | 296 |
| | 7 | Lentinus edodes(SHIITAKE) | 280 |
| Roasted with | 1 | Grifola umbellate(CHYOREIMAITAKE) | 799 |
| Far Infrared | 2 | Grifola frondosa(MAITAKE(BLACK)) | 768 |
| Ray + | ☆3 | Raw loquat seed | 621 |
| Fermentation | 4 | Agaricus blazei | 489 |
| with Koji | ★5 | Tricholoma matsutake(MATSUTAKE) | 412 |
| | 6 | Lentinus edodes(SHIITAKE) | 381 |
| | 7 | Pachyma hoelen(BUKURYO) | 334 |
| Roasted with | 1 | Grifola umbellate(CHYOREIMAITAKE) | 827 |
| Far Infrared | 2 | Grifola frondosa(MAITAKE(BLACK)) | 792 |
| Ray + | 3 | Raw loquat seed | 678 |
| Fermentation | ◯4 | Dried loquat seed | 572 |
| with Koji + | 5 | Agricus blazei | 509 |
| Oil-coat | 6 | Tricholoma matsutake(MATSUTAKE) | 430 |
| | 7 | Lentinus edodes(SHIITAKE) | 397 |
| | 8 | Pachyma hoelen(BUKURYO) | 362 |

Table 5 shows the comparison of MDA values for raw loquat seed containing amygdalin with that of individual mushroom. The mushroom used in Table 5 are:

tuckahoe (Pachyma hoelen), Agaricus blazei, Grifola umbellate(CHYOREI MAITAKE), Grifola frondosa (BLACK MAITAKE), Tricholoma matsutake (MATSUTAKE), Lentinus edodes (SHIITAKE) and Agaricus blazei was found to stronger express its antitumor activity by using the raw state fruity body than the dried material and upon using the raw state fruit body harvested is stored in refrigerator (4° C.) and use within 3 days.

Mushrooms other than Agaricus blazei in the case of using them in raw state showed no apparent efficacy difference by augmentation method when compared with those of crude drug supplied in dried state. Thus Tuckahoe (Pachyma hoelen), Grifola umbellate(CHYOREI MAITAKE), Grifola frondosa(BLACK MAITAKE), Tricholoma matsutake (MATSUTAKE), Lentinus edodes (SHIITAKE) used in this invention are dried materials.

Two kinds of Grifola frondosa (BLACK MAITAKE) are used. One is a product of Japan cultivated in Yukikuni, Niigata Prefecture, its formal name is Yukikunij MAITAKE or black MAITAKE, in this detailed description, it is described as “Black MAITAKE”. Another one is called as Chyorei MAITAKE(Grifola umbellate), collected from China. It is different from “Black MAITAKE”.

Anti-tumor activity of loquat seed can be relatively assessed by comparing its antitumor activity with those of antitumor mushrooms in Table 5.

For example, recently Agaricus blazei is attractive with its antitumor activity. The antitumor activity is higher for Agaricus blazei than that of raw loquat seed under not treatment condition.

The loquat seed only treated with roasting by far infrared ray radiation or with Koji fermentation, its antitumor activity was augmented to the level of Agricus blazei; and higher than that of tuckahoe, a kind of mushrooms said to possess the high antitumor activity. Furthermore, the loquat seed treated with the pairs of far infrared ray roasting+Koji fermentation and of infrared ray roasting+Koji fermentation+oil-coat, showed a higher antitumor activity, individually than Agaricus blazei.

The Kampo medicines use the natural crude drugs, among them, several rare mushrooms possessing high antitumor activity is supplied not enough. If use the above methods in this invention, the amygdalin-contained substance such as the loquat seed containing amygdalin which can be possibly and largely cultivated and an enough quantity can be harvested for and used as Kampo medicine instead of using such rare mushroom.

And this leads to the contribution of stable supply and low price of antitumor materials. Thus many patients with cancer may receive the efficacy of antitumor products.

In the above description, the most sophisticated form is to treat raw loquat seed with roasting, fermentation and oil-coat. But the antitumor activity of the raw loquat seed only treated with far infrared ray roasting or only fermentation with microorganisms such as Koji or both of roasting and fermentation, showed an increased expression of antitumor activity for treated loquat seed than that of non-treated loquat seed. In the case that oil-coat is not properly performed because of facility situation, only treating with far infrared ray roasting and fermentation with microorganism, their antitumor activity will be specially and effectively augmented than the non-treated loquat seed.

In the pairs of treatment: (1) far infrared ray roasting+fermentation+oil-coat or (2) far infrared ray roasting+fermentation, polysaccharide degrading enzyme treatment can be adopted instead of fermentation by using microorganisms such as Koji.

The sesame paste oil obtained from roasting sesame was used in oil-coat process. This contributes to augmentation method for antitumor activity.

The oil sources other than sesame, for example soybean, corn, red flower (Carthamus tinctoius L.), evening primrose, rice bran, rape seeds, etc. can be used as materials and oil obtained by roasting with far infrared ray and pressing.

To the medicinal composition and health food composition, if the loquat seed treated with an antitumor activity-augmented method consisting of roasting with far infrared ray, Koji fermentation and oil-coat were added, these compositions become effective for oral use of antitumor purpose.

In the product containing loquat seed treated with the augmentation method for antitumor, those compositions not affect the expression ability of antitumor effect, for example, vitamins, etc. can also be added in addition to the loquat seed. It also can ingredient with the anti-active oxygen substances developed by the present inventors to the products.

Composition containing the substance added with amygdalin treated with augment process for antitumor is better to supply as a gelatin capsule product, easy to oral administration. And proper medicinal additives, binding additives, etc. can also be used for tablet form product or granular product and pills.

Though this invention is the method of augmentation for antitumor activity in the case of oral administration. It is considered that if an injection drug prepared by adding the loquat seed treated with antitumor augmentation method, will contribute to expression ability of antitumor efficacy for the injection drug than the case of not use the augmentation method.

The above explanation is specially focused on the raw loquat seed containing amygdalin. There is not necessary to limit to the loquat seed. Seeds such as apricot, peach, plums, Japanese apricot, amygdaline peach, Amygdala amara and loquat leaf, alpha alpha, bamboo shoot, etc. can be used in this invention.

(EMBODIMENT 2)

In this embodiment, assessing method for efficacy of anti-cancer products such as loquat seed containing amygdalin and that for assessing antitumor efficacy of treatment of substance containing amygdalin are described.

As described in Practice Form 1, to the experimental system of peroxide lipid formation by ultra-violet ray, various product containing amygdalin-contained substance was added to determine the quantity of peroxide lipid for the assessment of antitumor activity.

The results show that the strong antitumor activity in oral administration, a large quantity of peroxide lipid formation is resulted with increasing the product concentration of loquat seed containing amygdalin.

Namely, the increased ratio of substance containing amygdalin can contribute to the higher ratio of peroxide lipid and this leads to strong expression of antitumor activity in oral route as so assessed.

In this connection, ranking of the antitumor finding ability can be decided by comparing various products containing amygdalin each other the increased ratio of peroxide lipid formed in response to the ratio of substance containing amygdalin added to the system for such complex products.

Two assessing methods according to the Example are explained as follows. The various treatment as shown in Table 1, when MDA values are compared to each other, MDA values for those samples received roasting with far infrared ray are high than untreated samples.

MDA value for sample treated with Koji fermentation is higher than the one of roasting with far infrared ray. MDA value for the sample treated with both roasting by far infrared ray and Koji fermentation processes, is found to be high than that for sample treated only with roasting or only with fermentation.

Furthermore, sample treated with far infrared ray roasting, Koji fermentation and oil-coat is found to be high than the sample treated with both roasting and fermentation.

On the other hand, efficacy rate of clinical trials on breast, stomach and lung cancers as shown in Tables 2, 3 and 4 are found to be in coincidence with high MDA values which come from treatments performed. This is to say, the high efficacy rate, the MDA values become high by treatment.

Antitumor activity finding ability can be so assessed that the more quantity formed for peroxide lipid by radiation docosahexaenoic acid with ultraviolet ray.

In connection with this, in Tables 2-4, by using the raw and dried seed, though “roasting with far infrared ray+Koji fermentation+oil-coat” pair treatment were applied, MDA values are low for dried seed and this is found to be in coincidence with low clinical efficacy rates.

This is to say that high value for MDA formed in the samples prepared in the system for peroxide lipid formation in which same concentration of substance containing amygdalin (raw and dried loquat seeds) treated with same processes showed a good finding ability for antitumor activity of treated seed.

Succeedingly, the higher value for MDA showing in the system of peroxide lipid formation by radiating the docosahexaenoic acid, the higher clinical efficacy rate, the assessment of antitumor activity for various substance containing amygdalin is high. Namely, there is a predicting method established for the efficacy assessment of antitumor activity of various substance containing amygdalin without performance of clinical trial.

The above explanation is done in the case of using loquat seed as the substance containing amygdalin. Seeds such as apricot, peach, plums, Japanese apricot, Chinese apricot, Amygdaline peach, Amygdala amara, and loquat leaf, alpha alpha bamboo shoot, etc. can be used in this invention.

Furthermore, in the above explanation docosahexaenoic acid is used. Those fatty acids other than docosahexaenoic acid such as biological unsaturated fatty acids, their formations of peroxide lipid quantity are affected by addition of substance containing amygdalin such as loquat seed, etc. can be used. And those unsaturated fatty acids other than biological body resources, their quantity of formation for peroxide lipid by radiation with ultra-violet ray are affected by addition of substances containing amygdalin, namely chemical synthesized fatty acids can also be used in the range of assessing methods.

By understanding the quantity of formation for peroxide lipid by radiation docosahexaenoic acid with ultraviolet ray in individual concentration of substance containing amygdalin added system, the mean and proper dose in response to the advanced stage of malignant tumor etc. can be possibly predicted without performance of clinical trial.

Namely, according to the points described above, by understanding the relationship of various concentration of amygdalin-contained substance with corresponding MDA values, the efficacy expression for each stage of cancer is proved in a previous clinical trail and surpass this on it, then MDA values become the efficacy parameter corresponding to clinical trial. The mean oral dose can be predicted by reversal calculation from concentration of substance containing amygdalin.

In this invention, comparison of antitumor activity for substance containing amygdalin can be performed by using common parameter: MDA values. Namely, the common dimension as the usable common parameter for various substance containing amygdalin, the item of MDA value has been established. And the relationship of MDA value with antitumor activity is also established. Thus assessment of antitumor activity can be performed each other.

In connection with this, screening of antitumor effect from many substances containing amygdalin can be searched in a short time by using this method.

According to the augmentation method of antitumor activity for substance containing amygdalin of this invention, the rate of efficacy in oral route is high for the treated loquat seed containing amygdalin than that of no treated loquat seed.

According to the augmentation method of antitumor activity for treated substance containing amygdalin can be augmented to be high than that or over that of mushrooms, substances not regarded as the substance containing amygdalin said to have the antitumor activity under not treated by these methods.

The product containing amygdalin-contained substances such as loquat seed, treated with augmentation method of antitumor, showed a high rate of efficacy of antitumor activity in oral route than the untreated product containing amygdalin-contained substance.

According to method for assessing antitumor efficacy of treatment for amygdalin contained substance according to the present invention, efficacy in view of the antitumor activity by the various kinds of treatment methods applied to the amygdalin-contained substance can be simply assessed within a short term.

According to the assessment method of efficacy for antitumor activity of substance containing amygdalin can be predicted not through the oral route clinical trial. For this reason when compared with the conventional methods: after long-term animal tests and clinical trail, the results are finally obtained. By using the invented methods to screen for active antitumor enormous substances of natural products can be searched simple than the conventional methods.

What is claimed is:

1. An Augmentation method for antitumor activity of substances containing amygdalin, which comprises a roasting process of roasting the substances containing amygdalin with far infrared ray and a fermentation process of fermenting with microorganism to augment the antitumor activity of the substances containing amygdalin than the case without the roasting and the fermentation.

2. The augmentation method for antitumor activity of substances containing amygdalin according to claim 1, wherein
succeeding to the fermentation process, an oil-coat process in which the substances after the fermentation in the fermentation process is subjected to coat with the oil obtained from plants such as sesame roasted with far infrared ray.

3. The augmentation method for antitumor activity of substances containing amygdalin according to claim 1 or 2, wherein
the substances containing amygdalin is a amygdalin-contained plant seed.

4. The augmentation method for antitumor activity of substances containing amygdalin according to claim 3, wherein
the plant seed is loquat seed.

5. The augmentation method for antitumor activity of substances containing amygdalin according to claim 4, wherein
the loquat seed is roasted in the raw state with far infrared ray.

6. An antitumor activity augmented amygdalin contained substance-containing composition to which the augmentation method for antitumor activity of substances containing amygdalin is applied, the said method comprises a roasting process of roasting the substances containing amygdalin with far infrared ray and a fermentation process of fermenting with microorganism to augment the antitumor activity of the substances containing amygdalin than the case without the roasting and the fermentation.

7. The antitumor activity augmented amygdalin-contained substance containing composition according to claim 6, wherein
succeeding to the fermentation process, an oil-coat process in which the substances after the fermentation in the fermentation process is subjected to coat with the oil obtained from plants such as sesame roasted with far infared ray.

8. The antitumor activity augmented amygdalin-contained substance containing composition according to claim 6 or 7, wherein
the substances containing amygdalin is amygdalin-contained plant seed.

9. The antitumor activity augmented amygdalin-contained substance-containing composition according to claim 8, wherein
the plant seed is loquat seed.

10. The antitumor activity augmented amygdalin-contained substance containing composition according to claim 9, wherein
the loquat seed is roasted in raw state with far infrared ray.

11. The assessment method of efficacy of treatment to be applied to the substances containing amygdalin, which comprises adding the substances containing amygdalin to which the treatment is applied to a system of peroxide lipid formation by radiation to an unsaturated fatty acid such as docosahexaenoic acid with ultraviolet ray and assessing an efficacy of treatment for augmenting the antitumor activity of the substances containing amygdalin to be large as an increased ratio of formed amount of the peroxide lipid to an increased ratio of the concentration of substances containing amygdalin being large.

12. The assessment method of efficacy of treatment to be applied to the substances containing amygdalin according to claim 11, wherein
the substance containing amygdalin is amygdalin-contained plant seed.

13. The assessment method of efficacy of treatment to be applied to the substances containing amygdalin according to claim 12, wherein
the plant seed is loquat seed.

14. The assessment method of efficacy of treatment to be applied to the substances containing amygdalin according to claim 13, wherein
the loquat seed is roasted in the raw state with far infrared ray.

15. An Assessment method not through clinical trial, antitumor efficacy for amygdalin-contained substances, which comprises adding the substance containing amygdalin to a system of peroxide lipid formation by radiation to an unsaturated fatty acid such as docosahexaenoic acid with ultraviolet ray and assessing antitumor efficacy of substances containing amygdalin to be large in oral administration as an increase ratio of formed amount of the peroxide lipid to an increased ratio of the substances containing amygdalin being large.

16. The assessment method for antitumor efficacy of amygdalin-contained substance according to claim 15, wherein
the augmentation method for antitumor activity of substances containing amygdalin is applied to the amygdalin-contained substance, the said method comprises a roasting process of roasting the substances containing amygdalin with far infrared ray and a fermentation with microorganism to augment the antitumor activity of the substances containing amygdalin than the case without the roasting and the fermentation.

17. The assessment method for antitumor efficacy of amygdalin-contained substance according to claim 16, wherein
succeeding to the fermentation process, an oil-coat process in which the substances after the fermentation in fermentation process is subjected to coat with the oil obtained from plants such as sesame roasted with far infrared ray.

18. The assessment method for antitumor efficacy of amygdalin-contained substance according to claim 16 or 17, wherein
the amygdalin-contained substance is amygdalin-contained plant seed.

19. The assessment method for antitumor efficacy of amygdalin-contained Substance according to claim 18, wherein
the plant seed is loquat seed.

20. The assessment method for antitumor efficacy of amygdalin-contained substance according to claim 19, wherein
the loquat seed is roasted in the raw state with far infrared ray.
